(19) United States
(12) Patent Application Publication
(10) Pub. No.: US 2025/0127927 A1
(43) Pub. Date: Apr. 24, 2025

(54) TARGETED INTEGRATION AT BETA-GLOBIN LOCUS IN HUMAN HEMATOPOIETIC STEM AND PROGENITOR CELLS

(71) Applicants: The Board of Trustees of the Leland Stanford Junior University, Stanford, CA (US); The Regents of the University of California, Oakland, CA (US); The J.David Glastone Institutes,A Testamentary Trust Established under the Will of J.David Gladstone, San Francisco, CA (US)

(72) Inventors: Matthew H. Porteus, Stanford, CA (US); Michael Kyle Cromer, Stanford, CA (US); Tippi MacKenzie, San Francisco, CA (US); Beeke Wienert, San Francisco, CA (US)

(73) Assignees: The Board of Trustees of the Leland Stanford Junior University, Stanford, CA (US); The Regents of the University of California, Oakland, CA (US)

(21) Appl. No.: 18/685,648

(22) PCT Filed: Aug. 23, 2022
(86) PCT No.: PCT/US2022/075313

§ 371 Date: Feb. 22, 2024

Related U.S. Application Data
(60) Provisional application No. 63/236,178, filed on Aug. 23, 2021.

(51) Int. Cl. A61K 48/00; C12N 9/22; C12N 15/11; C12N 15/90

(52) U.S. Cl. CPC A61K 48/0058; C12N 9/22; C12N 15/11; C12N 15/907; C12N 2310/20; C12N 2800/80

(57) ABSTRACT

The present disclosure provides methods and compositions for genetically modifying hematopoietic stem and progenitor cells (HSPCs), in particular by replacing the HBB locus in the HSPCs with a transgene encoding alpha globin.

### CROSS REFERENCE TO RELATED APPLICATIONS

The present application claims priority to U.S. Provisional Pat. Appl. No. 63/236,178, filed on Aug. 23, 2021, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under Grant No. HL135607 awarded by the National Institutes of Health. The Government has certain rights in the invention.

### BACKGROUND

α-thalassemia is one of the most common autosomal recessive disorders in the world. Severe forms are estimated to occur in about 1 in 1,000,000 individuals in North America. α-thalassemia is caused by a reduction or absence of alpha-globin subunits, resulting in a loss of functional hemoglobin in red blood cells. Symptoms of α-thalassemia can vary substantially, in part due to the number of affected alpha-globin genes in an individual. For example, individuals with a single mutation in one copy of HBA1 or HBA2 (i.e., silent carriers) have no symptoms, individuals with mutations in two copies (either one copy of HBA1 and one copy of HBA2, or both copies of either HBA1 or HBA2) (i.e., alpha thalassemia minor) are generally asymptomatic or only have very mild symptoms, individuals with mutations in three copies (HbH disease) have sometimes severe anemia, with microcytic red blood cells and low levels of functional hemoglobin, and individuals with no functional alpha globin genes (Hb Bart's hydrops fetalis) rarely survive until birth or shortly thereafter.

The current standard of care for α-thalassemia involves frequent blood transfusions combined with iron chelation therapy. Currently, the only curative strategy for this disease is allogeneic hematopoietic stem cell transplantation (HSCT) from an immunologically matched donor. However, in the majority of cases no matched donor is available for allogeneic HSCT, and, even if one is identified, transplants from these donors carry a risk of immune rejection and graft-versus-host disease.

A potentially ideal treatment would involve isolation of patient-derived hematopoietic stem and progenitor cells (HSPCs), introduction of HBA1 or HBA2 to restore alpha globin protein levels, followed by autologous HSCT of the patient's own corrected HSPCs, which would carry no risk of immune rejection. Such approaches, however, face numerous substantial safety and efficacy issues, and do not necessarily address the genetic cause of α-thalassemia-inactivation of HBA1 and/or HBA2—and may not sufficiently rescue the disease phenotype in vivo. Furthermore, these therapies act to compensate only for the lack of HBA, and do not diminish levels of β-globin.

There is thus a need for new, safe and effective approaches for introducing HBA1 or HBA2 transgenes into autologous HSPCs and red blood cells in vivo or ex vivo, increasing the amount of alpha globin in red blood cells and improving the balance between alpha and beta globin. The present disclosure satisfies this need and provides other advantages as well.

### BRIEF SUMMARY

In one aspect, the present disclosure provides a method of genetically modifying a hematopoietic stem and progenitor cell (HSPC) from a subject, the method comprising: introducing into the HSPC a guide RNA comprising a sequence that hybridizes to an intron of an HBB gene sequence, an RNA-guided nuclease, and a donor template comprising a transgene encoding an α-globin protein, wherein the donor template comprises a first homology arm located 5′ of the transgene, the first homology arm corresponding to at least 200 nucleotides of the HBB gene sequence beginning at the start codon and continuing upstream thereof, and a second homology arm located 3′ of the transgene, the second homology arm corresponding to at least 200 nucleotides of the HBB gene sequence, beginning at the guide RNA target site and continuing downstream thereof; wherein the RNA-guided nuclease cleaves the intron of the HBB gene sequence in the cell and the transgene is integrated into the cleaved HBB gene sequence; thereby generating a genetically modified HSPC; wherein the integrated transgene results in expression of the α-globin protein in the genetically modified HSPC.

In some embodiments, the method further comprises isolating the HSPC from the subject prior to introducing the guide RNA, the RNA-guided nuclease, and the donor template. In some embodiments, the first homology arm comprises at least about 300, 400, 500, 600, 700, 800, 900, or more nucleotides. In some embodiments, the first homology arm comprises the nucleotide sequence of SEQ ID NO: 1 or a subsequence thereof, or a sequence comprising at least about 80%, 85%, 90%, 95%, 96%, 97%, 98%, 99%, or more identity to SEQ ID NO:1 or a subsequence thereof. In some embodiments, the second homology arm comprises at least about 300, 400, 500, 600, 700, 800, 900, or more nucleotides. In some embodiments, the second homology arm comprises the nucleotide sequence of SEQ ID NO: 2 or a subsequence thereof, or a sequence comprising at least about 80%, 85%, 90%, 95%, 96%, 97%, 98%, 99%, or more identity to SEQ ID NO:2 or a subsequence thereof. In some embodiments, the intron of the HBB gene sequence is intron 1.

In some embodiments, the target sequence of the guide RNA comprises the nucleotide sequence of SEQ ID NO:14. In some embodiments, the intron of the HBB gene sequence is intron 2. In some embodiments, the target sequence of the guide RNA comprises the nucleotide sequence of SEQ ID NO:15 or SEQ ID NO:16. In some embodiments, the guide RNA comprises one or more 2′-O-methyl-3′-phosphorothioate (MS) modifications. In some embodiments the one or more 2′-O-methyl-3′-phosphorothioate (MS) modifications are present at the three terminal nucleotides of the 5′ and 3′ ends of the guide RNA. In some embodiments, the RNA-guided nuclease is Cas9. In some embodiments, the guide RNA and the RNA-guided nuclease are introduced into the HSPC as a ribonucleoprotein (RNP) complex by electroporation.

In some embodiments, the transgene is an HBA1 transgene. In some such embodiments, the transgene comprises the nucleotide sequence of SEQ ID NO:4, or a sequence comprising at least about 80%, 85%, 90%, 95%, 96%, 97%, 98%, 99%, or more identity to SEQ ID NO:4. In some embodiments, the transgene is an HBA2 transgene. In some such embodiments, the transgene comprises the nucleotide sequence of SEQ ID NO:5, or a sequence comprising at least about 80%, 85%, 90%, 95%, 96%, 97%, 98%, 99%, or more identity to SEQ ID NO:5. In some embodiments, the expression of the transgene is driven by an endogenous HBB promoter. In some embodiments, the transgene comprises a 5′ UTR derived from the HBB gene. In some embodiments, the 5′ UTR comprises the nucleotide sequence of SEQ ID NO:7. In some embodiments, the transgene comprises a 5′ UTR derived from the HBA1 or HBA2 gene. In some embodiments, the 5′ UTR comprises the nucleotide sequence of SEQ ID NO:9. In some embodiments, the transgene comprises a 3′ UTR derived from the HBB gene. In some embodiments, the 5′ UTR comprises the nucleotide sequence of SEQ ID NO:8. In some embodiments, the transgene comprises a 3′ UTR derived from the HBA1 or HBA2 gene. In some embodiments, the 3′ UTR comprises the nucleotide sequence of SEQ ID NO:10 or SEQ ID NO:11. In some embodiments, the donor template is introduced into the HSPC using a recombinant adeno-associated virus (rAAV) vector. In some embodiments, the rAAV vector is a AAV6 vector.

In some embodiments, the HSPC comprises an HBA1 or HBA2 gene that comprises a mutation or deletion as compared to a wild-type HBA1 or HBA2 gene. In some embodiments, the mutation is causative of a disease. In some embodiments, the disease is alpha-thalassemia. In some embodiments, the method increases the level of adult hemoglobin tetramers in the HSPC as compared to prior to introduction of the guide RNA, the RNA-guided nuclease, and the donor template. In some embodiments, the subject has alpha-thalassemia, and wherein the genetically modified HSPC is reintroduced into the subject. In some embodiments, the reintroduction of the genetically modified HSPC ameliorates one or more symptoms of the alpha-thalassemia. In some embodiments, the subject is a human. In some embodiments, the method comprises genetically modifying a population of HSPCs from the subject.

In another aspect, the present disclosure provides a genetically modified HSPC comprising an HBA1 or HBA2 transgene integrated in an HBB locus, wherein the genetically modified HSPC is generated using any of the herein-disclosed methods.

In some embodiments, the HBA1 or HBA2 transgene has replaced an endogenous HBB coding sequence in the genome of the genetically modified HSPC.

In a related aspect, the present disclosure provides a population of HSPCs comprising the genetically modified HSPC described herein.

In another aspect, the present disclosure provides a method for treating alpha-thalassemia in a subject in need thereof, the method comprising administering any of the herein-described genetically modified HSPCs or a population thereof to the subject, wherein the genetically modified HSPC engrafts in the subject and results in an increased level of adult hemoglobin tetramers in the subject as compared to prior to the administration, thereby treating alpha-thalassemia in the subject.

### BRIEF DESCRIPTION OF THE DRAWINGS

FIGS. 1A-1B: Integrating α-globin at HBB without disrupting β-globin. Custom integrations using CRISPR/AAV6-mediated genome editing are achieved when cleavage is initiated by a single Cas9 gRNA followed by transduction with an AAV6 DNA repair template with a right homology arm that corresponds to the ˜900 bp immediately downstream of the Cas9 cut site and a left homology arm that is split off from the cut site to correspond to the ˜900 bp immediately upstream of the start codon of the endogenous gene. This strategy is capable of mediating a high frequency of recombination in primary human CD34+ HSPCs. FIG. 1A: 3′UTR integration with a split left homology arm (LHA). FIG. 1B: Intron integration with a split left homology arm.

FIG. 2: Custom Cas9 gRNAs were screened for their efficacy in the human HUDEP-2 cell line by pre-complexing Cas9 protein with each respective gRNA and delivering the complexes by electroporation. 2-4 d post-targeting, genomic DNA was harvested and subjected to PCR amplification of the region surrounding the expected cut site and resulting Sanger sequences were analyzed for indel frequencies using the online ICE tool from Synthego.

FIGS. 3A-3E: AAV vectors mediate integration into the start codon of HBB. AAV6 DNA repair templates were designed, cloned, produced, and purified with a right homology arm that corresponds to the ˜900 bp immediately downstream of the Cas9 cut site and a left homology arm that is split off from the cut site to correspond to the ˜900 bp immediately upstream of the start codon of the endogenous gene. Templates were designed for use with guide RNAs targeting intron 1 (e.g., gRNA 7) (FIG. 3A) or targeting intron 2 (e.g., gRNAs 11 and 13) (FIG. 3B). FIG. 3C: Primary human CD34+-enriched HSPCs were plated and expanded in HSC expansion media, and then two days later high-fidelity Cas9 protein pre-complexed with either sgRNA 7, 11, or 13 was delivered via electroporation. The HSPCs were then immediately transduced with the AAV6 DNA repair donor corresponding to intron 1 or intron 2 integration schemes. Five days later, the percentage of GFP+ cells was determined by flow cytometry. FIGS. 3D-3E: The intron 1 integration scheme was found to be more efficient (FIG. 3D) and yield higher mean fluorescence intensities (MFIs) per edited cell (FIG. 3E) than either intron 2 integration scheme.

FIGS. 4A-4E: α-globin transgenes integrate with an RBC-specific expression profile at the HBB locus. Promoterless HBA-2A-YFP integration cassettes packaged in AAV6 vectors were designed, cloned, produced, and purified. Primary human CD34+ HSPCs were then plated, expanded, and edited using sgRNA 7 and transduction with the promoterless repair vectors displayed (FIG. 4A). Because HBB was not expected to be expressed until HSPCs had begun erythroid differentiation, we used a three-liquid phase erythroid differentiation protocol and measured fluorescence by flow cytometry at day 18 post-editing (FIG. 4B). The percentage of red blood cells (FIG. 4C) were quantified for each of the vectors from FIG. 4A, along with the percentages of GFP+ cells (FIG. 4D) and the mean fluorescence intensities (MFIs) from among the GFP+ cells (FIG. 4E).

FIGS. 5A-5B: Expression kinetics were tracked by measuring fluorescence by flow cytometry during the course of erythroid differentiation. All editing conditions differentiate at approximately the same rate (FIG. 5A), and fluorescence from promoterless HBA-2A-YFP constructs begins to occur around day 6 and plateaus around day 12 (FIG. 5B).

FIG. 6: The percentage of YFP+ cells was plotted at day 18 post-editing (at the end of erythroid differentiation). No fluorescence was detected above background in CD45+/CD71−/GPA− HSPCs, but a high degree of fluorescence was observed in CD45−/CD71+/GPA+ red blood cells.

FIGS. 7A-7C: Promoterless HBA1 AAV6 repair templates were designed with HBB and HBA1 UTRs, but with the 2A-YFP removed (FIG. 7A). These translational vectors were first tested in WT primary human CD34+ HSPCs for their ability to efficiently integrate (FIG. 7B), and were also tested in alpha thalassemia patient-derived CD34+ HSPCs (FIG. 7C). FIGS. 7B and 7C show the percentage of red blood cells (% RBCs; left panels) and the percentage of edited alleles (right panels) for each of the vectors from FIG. 7A.

FIG. 8: Cassettes have also been designed to integrate HBG1 (gamma-globin) along with the HBA1 transgene. These vectors use sgRNA 7, intron 1 homology arms, and HBA1 UTRs. Linking these two transgenes will be a Furin-P2A cleavage peptide sequence to minimize the possibility that the corresponding cleavage tails at the C-terminus and N-terminus of the two transgenes will disrupt hemoglobin tetramer formation.

### DETAILED DESCRIPTION

### 1. Introduction

The present disclosure provides methods and compositions for integrating transgenes, e.g., for alpha-globin genes such as HBA1 or HBA2, into an intron of the HBB locus in hematopoietic stem and progenitor cells (HSPCs).

The present methods can be used to introduce alpha-globin transgenes, e.g., coding sequences with optional elements such as promoters or other regulatory elements (e.g., enhancers, repressor domains), introns, WPREs, poly A regions, UTRs (e.g. 3′ UTRs), specifically into an intron or other non-coding region of the HBB locus of HSPCs. In particular, the present disclosure provides guide RNA sequences that specifically recognize sites within HBB introns or other non-coding sequences, enabling the cleavage of HBB by an RNA-directed nuclease such as Cas9 in a fashion such that the coding sequence of HBB is left intact; accordingly, a cleavage event that occurs at an HBB locus, but in the absence of homology-directed repair using any of the herein-described donor repair templates, does not render the gene non-functional and disrupt the expression of β-globin from the locus. By cleaving HBB in the presence of a donor template comprising a transgene, the transgene can integrate into the genome at the site of cleavage by homology directed recombination (HDR), e.g., replacing the endogenous HBB gene. Further, in particular embodiments, the homology arms in the donor templates used in the present methods are split, i.e., non-contiguous, in that the left arm starts at or around the translation start site and runs upstream, and the right arm starts at or around the cleavage site and rund downstream.

In particular embodiments, the present methods can be used to deliver an HBA (i.e., HBA1 or HBA2) transgene into the HBB locus, which could be used as a universal treatment strategy for patients with α-thalassemia, regardless of which mutations in HBA1 or HBA2 are responsible for the disease. In particular, integration at this locus is able to produce high levels of functional transgene, capable of forming adult hemoglobin tetramers, and to reduce the amount of beta globin, thereby restoring the balance between the alpha and beta globin chains.

### 2. General

Practicing this disclosure utilizes routine techniques in the field of molecular biology. Basic texts disclosing the general methods of use in this disclosure include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255:137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of an HBA1 or HBA2 cDNA, transgene, or encoded protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215:403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” or “donor template” refers to a polynucleotide sequence that can be used to repair a double stranded break (DSB) in the DNA, e.g., a CRISPR/Cas9-mediated break at an HBB locus as induced using the herein-described methods and compositions. The homologous repair template comprises homology to the genomic sequence surrounding the DSB, i.e., comprising HBB homology arms. In particular embodiments, two distinct homologous regions are present on the template, with each region comprising at least 50, 100, 200, 300, 400, 500, 600, 700, 800, 900, 400-1000, 500-900, or more nucleotides of homology with the corresponding genomic sequence. In particular embodiments, the templates comprise two homology arms comprising, e.g., about 900 nucleotides of homology, with one arm extending upstream starting at the translation start site, and the other arm extending downstream from the sgRNA target site. The repair template can be present in any form, e.g., on a plasmid that is introduced into the cell, as a free floating doubled stranded DNA template (e.g., a template that is liberated from a plasmid in the cell), or as single stranded DNA. In particular embodiments, the template is present within a viral vector, e.g., an adeno-associated viral vector such as AAV6. The templates of the present disclosure can also comprise a transgene, e.g., HBA1 or HBA2 transgene.

As used herein, “homologous recombination” or “HR” refers to insertion of a nucleotide sequence during repair of double-strand breaks in DNA via homology-directed repair mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments, HR involves double-stranded breaks induced by CRISPR-Cas9.

HBA1 and HBA2 (hemoglobin subunit alpha 1 and 2, respectively) are closely related, but not identical, genes encoding alpha-globin, which is a component of hemoglobin. HBA1 and HBA2 are located within the alpha-globin locus, located on human chromosome 16. Their coding sequences are identical, but the genes diverge, e.g., in the 5′UTRs, introns, and particularly the 3′UTRs. The NCBI gene ID for human HBA1 is 3039, the NCBI gene ID for human HBA2 is 3040, and the UniProt ID for human alpha-globin is V9H1D9, the entire disclosures of which are herein incorporated by reference. An exemplary HBA1 transgene is shown herein as SEQ ID NO:4, and an exemplary HBA2 transgene is shown herein as SEQ ID NO: 5. The amino acid sequence of human alpha-globin is shown herein as SEQ ID NO: 17. As used herein, HBA1 can refer to any nucleotide sequence comprising about 70%, 75%, 80%, 85%, 90%, 95%, 96%, 97%, 98%, 99%, or more homology to SEQ ID NO:4 or a subsequence therein, and HBA2 can refer to any nucleotide sequence comprising about 70%, 75%, 80%, 85%, 90%, 95%, 96%, 97%, 98%, 99%, or more homology to SEQ ID NO:5 or a subsequence therein. “HBA” can refer to either HBA1 or HBA2, or to any nucleotide sequence encoding the alpha globin protein, i.e., SEQ ID NO:17, or a fragment thereof, or any protein comprising at least about 70%, 75%, 80%, 85%, 90%, 95%, 96%, 97%, 98%, 99%, or more homology to SEQ ID NO: 17 or a fragment thereof.

HBB (hemoglobin subunit beta) is a gene encoding the beta subunit of hemoglobin, which in normal adults comprises two alpha chains and two beta chains. Mutations in HBB, e.g., causing a reduction or absence of HBB expression or function, can cause β-thalassemia. The NCBI gene ID No. for human HBB is 3043, and the UniProt ID is P68871, the entire disclosures of which are herein incorporated by reference. In humans, the HBB locus is located on chromosome 11 (11p15.4).

HBG1 and HBG2 are genes encoding gamma globin, which is normally expressed in the fetal liver, spleen, and bone marrow. Gamma globin forms part of fetal hemoglobin (HbF), which comprises two gamma globin chains and two alpha globin chains. The NCBI gene ID NO. for human HBG1 is 3047, the NCBI gene ID NO: for human HBG2 is 3048, the UniProt ID for human gamma-globin 1 (the product of HBG1) is P69891, and the UniProtID for human gamma-globin 2 (the product of HBG2) is P69892, the entire disclosures of which are herein incorporated by reference. “HBG” can refer to either HBG1 or HBG2, or to a polynucleotide encoding gamma globin 1 or gamma globin 2.

“Alpha thalassemia” or “α-thalassemia” refers to an inherited disease caused by a deficiency in alpha globin, a component of hemoglobin. Adult hemoglobin is a tetramer that primarily consists of two alpha and two beta globin molecules. Individuals carry four alpha-globin gene copies (two copies of HBA1 and two copies of HBA2), and alpha thalassemia can be caused, e.g., by deletion or mutation of one or more of these genes. In some embodiments, the subject lacks two copies, and in particular embodiments the subject lacks three or four functional alpha-globin genes. Symptoms can be caused in part by an imbalance between alpha and beta globin proteins, resulting in, e.g., the formation of hemoglobin H, a tetramer formed from four beta chains. Alpha thalassemia can range in severity, from no symptoms (e.g., in silent carriers) to mild symptoms (in carriers with two defective copies), to moderate to severe hemolytic anemia (three defective copies) to severe anemia that is usually fatal (alpha thalassemia major, or no functional copies). The present methods can be used to treat any form of alpha thalassemia, regardless of the particular HBA1 or HBA2 genes that are deficient in the patient and regardless of the nature of the HBA1 and/or HBA2 mutations causing the disease.

As used herein, the terms “hematopoietic stem and progenitor cell” and “HSPC” refer to a hematopoietic stem cell (HSC), a hematopoietic progenitor cell (HPC), or a population of hematopoietic stem cells and hematopoietic progenitor cells.

### 4. CRISPR/Cas Systems Specifically Targeting the HBB Locus

The present disclosure is based in part on the identification of CRISPR guide sequences that specifically direct the cleavage of HBB by RNA-guided nucleases, in particular within non-coding sequences such as an intron or 3′ untranslated region (UTR). In particular embodiments, the guide RNA targets an intron of HBB, e.g., intron 1. The present disclosure provides a CRISPR/AAV6-mediated genome editing method that can achieve high rates of targeted integration at the HBB locus. The integrated alpha-globin encoding transgenes exhibit RBC-specific expression of alpha globin, and cells edited at this locus are capable of long-term engraftment and hematopoietic reconstitution.

Because of the targeting of non-coding regions such as introns, cleavage by the RNA-guided nuclease at the sgRNA target site can lead, e.g., to integration of an HBA transgene at the HBB locus, or to the possible production of indels at the site of cleavage that does not disrupt the expression of the HBB gene. As such, in a substantial number of cells one copy of the HBB gene will be replaced by an HBA transgene, and the coding sequence of other HBB gene will be left intact and still capable of expressing beta-globin. Furthermore, in the treatment of α-thalassemia, because the pathology is caused both by lack of HBA (i.e., HBA1 or HBA2) as well as aggregation of unpaired beta-globin, knocking HBA into HBB addresses both problems in a single genome editing event, allowing the simultaneous increase of HBA levels and decrease of levels of beta-globin.

sgRNAs

The single guide RNAs (sgRNAs) of the present disclosure target the HBB locus, in particular within a non-coding region such as an intron. In particular embodiments, the sgRNA targets intron 1 of the HBB gene. sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at the HBB locus, and a constant region that mediates binding to Cas9 or another RNA-guided nuclease. The sgRNA can target any sequence within HBB adjacent to a PAM sequence. In particular embodiments, the sgRNA targets a sequence within an intron or other non-coding sequence within HBB that is adjacent to a PAM sequence. In particular embodiments, the sgRNA targets a sequence within HBB intron 1. In some embodiments, the target sequence comprises one of the sequences shown as SEQ ID NOS: 14-16, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions, or subtractions relative to, one of SEQ ID NOS: 14-16. In particular embodiments, the target sequence comprises the target sequence of sg7 (SEQ ID NO:14), or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to, SEQ ID NO:7.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgRNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g, 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′-O-methyl-3′-phosphoacetate modifications, 2′-fluoro-pyrimidines, S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33 (9): 985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA as described herein and being guided to and cleaving the specific HBB sequence targeted by the targeting sequence of the sgRNA. In particular embodiments, the Cas9 is from Streptococcus pyogenes.

Also disclosed herein are CRISPR/Cas or CRISPR/Cpf1 systems that target and cleave DNA at the HBB locus. An exemplary CRISPR/Cas system comprises (a) a Cas (e.g., Cas9) or Cpf1 polypeptide or a nucleic acid encoding said polypeptide, and (b) an sgRNA that hybridizes specifically to HBB, (e.g., an HBB intron), or a nucleic acid encoding said guide RNA. In some instances, the nuclease systems described herein, further comprises a donor template as described herein. In particular embodiments, the CRISPR/Cas system comprises an RNP comprising an sgRNA targeting HBB and a Cas protein such as Cas9.

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the HBB locus to carry out the methods disclosed herein.

Introducing the sgRNA and Cas Protein into Cells

The guide RNA and nuclease can be introduced into the cell using any suitable method, e.g., by introducing one or more polynucleotides encoding the guide RNA and the nuclease into the cell, e.g., using a vector such as a viral vector or delivered as naked DNA or RNA, such that the guide RNA and nuclease are expressed in the cell. In some embodiments, one or more polynucleotides encoding the sgRNA, the nuclease or a combination thereof are included in an expression cassette. In some embodiments, the sgRNA, the nuclease, or both sgRNA and nuclease are expressed in the cell from an expression cassette. In some embodiments, the sgRNA, the nuclease, or both sgRNA and nuclease are expressed in the cell under the control of a heterologous promoter. In some embodiments, one or more polynucleotides encoding the sgRNA and the nuclease are operatively linked to a heterologous promoter. In particular embodiments, the guide RNA and nuclease are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the RNA-binding nuclease (e.g., Cas9) assembled with the guide RNA (e.g., sgRNA), such that the RNPs are capable of binding to the target DNA (through the gRNA component of the RNP) and cleaving it (via the protein nuclease component of the RNP). As used herein, an RNP for use in the present methods can comprise any of the herein-described guide RNAs and any of the herein-described RNA-guided nucleases.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cells are CD34+ hematopoietic stem and progenitor cells (HSPCs), e.g., cord blood-derived (CB), adult peripheral blood-derived (PB), or bone marrow derived HSPCs.

HSPCs can be isolated from a subject, e.g., by collecting mobilized peripheral blood and then enriching the HSPCs using the CD34 marker. In some embodiments, the cells are from a subject with α-thalassemia. In such embodiments, the transgene that is integrated into the genome of the HSPC is HBA1. In such embodiments, the transgene that is integrated into the genome of the HSPC is HBA2. In some embodiments, a method is provided of treating a subject with α-thalassemia, comprising genetically modifying a plurality of HSPCs isolated from the subject so as to integrate an HBA1 or HBA2 transgene at the HBB locus (e.g., an intron within the HBB locus), and reintroducing the HSPCs into the subject. In some such embodiments, HSPCs differentiate into red blood cells (RBCs) in vivo, and the RBCs express higher levels of alpha-globin, and lower levels of beta-globin, as compared to the levels in RBCs from the subject that have not been subjected to the present methods.

To avoid immune rejection of the modified cells when administered to a subject, the cells to be modified are preferably derived from the subject's own cells. Thus, preferably the mammalian cells are autologous cells from the subject to be treated with the modified cells. In some embodiments, however, the cells are allogeneic, i.e., isolated from an HLA-matched or HLA-compatible, or otherwise suitable, donor.

In some embodiments, cells are harvested from the subject and modified according to the methods disclosed herein, which can include selecting certain cell types, optionally expanding the cells and optionally culturing the cells, and which can additionally include selecting cells that contain the transgene integrated into the HBB locus. In particular embodiments, such modified cells are then reintroduced into the subject.

Further disclosed herein are methods of using said nuclease systems to produce the modified host cells described herein, comprising introducing into the cell (a) an RNP of the present disclosure that targets and cleaves DNA at the HBB locus, and (b) a homologous donor template or vector as described herein. Each component can be introduced into the cell directly or can be expressed in the cell by introducing a nucleic acid encoding the components of said one or more nuclease systems.

Such methods will target integration of the functional transgene, e.g., HBA1 or HBA2 transgene, at the endogenous HBB locus in a host cell ex vivo. Such methods can further comprise (a) introducing a donor template or vector into the cell, optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA specific to the HBB locus, and (b) a homologous donor template or vector as described herein.

In any of these methods, the nuclease can produce one or more single stranded breaks within the HBB locus, or a double-stranded break within the HBB locus. In these methods, the HBB locus is modified by homologous recombination with said donor template or vector to result in insertion of the transgene into the locus. The methods can further comprise (c) selecting cells that contain the transgene integrated into the HBB locus.

In some embodiments, i53 (Canny et al. (2018) Nat Biotechnol 36:95) is introduced into the cell in order to promote integration of the donor template by homology directed repair (HDR) versus integration by non-homologous end-joining (NHEJ). For example, an mRNA encoding i53 can be introduced into the cell, e.g., by electroporation at the same time as an sgRNA-Cas9 RNP. The sequence of i53 can be found, inter alia, at www.addgene.org/92170/sequences/.

Techniques for the insertion of transgenes, including large transgenes, capable of expressing functional proteins, including enzymes, cytokines, antibodies, and cell surface receptors are known in the art (See, e.g. Bak and Porteus, Cell Rep. 2017 Jul. 18; 20 (3): 750-756 (integration of EGFR); Kanojia et al., Stem Cells. 2015 October; 33 (10): 2985-94 (expression of anti-Her2 antibody); Eyquem et al., Nature. 2017 Mar. 2; 543 (7643): 113-117 (site-specific integration of a CAR); O'Connell et al., 2010 PLOS ONE 5 (8): e12009 (expression of human IL-7); Tuszynski et al., Nat Med. 2005 May; 11 (5): 551-5 (expression of NGF in fibroblasts); Sessa et al., Lancet. 2016 Jul. 30; 388 (10043): 476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016:539, 384-389 (adding tNGFR into hematopoietic stem cells (HSC) and HSPCs to select and enrich for modified cells); each of which is herein incorporated by reference in its entirety.

### Homologous Repair Templates

The transgene to be integrated, which is comprised by a polynucleotide or donor construct, can be any alpha globin transgene whose gene product can provide functional alpha globin in red blood cells. For example, the transgene could be used to replace or compensate for a defective gene, e.g., a defective HBA1 or HBA2 gene in a subject with α-thalassemia. In particular embodiments, the HBA1 or HBA2 transgene in the homologous repair template comprises SEQ ID NO:4 or SEQ ID NO:5 or a subsequence of SEQ ID NO:4 or SEQ ID NO:5, or a sequence comprising at least about 70%, 75%, 80%, 85%, 90%, 95%, 96%, 97%, 98%, 99%, or more identity to SEQ ID NO:4 or SEQ ID NO:5 or a subsequence of SEQ ID NO:4 or SEQ ID NO:5.

In some embodiments, the donor template will also comprise a gamma-globin-encoding transgene, e.g., an HBG1 or HBG2 transgene. In some embodiments, the HBG transgene is an HBG1 transgene, e.g., comprising the sequence shown as SEQ ID NO:12 or a subsequence thereof, or a sequence comprising at least about 70%, 75%, 80%, 85%, 90%, 95%, 96%, 97%, 98%, 99%, or more identity to SEQ ID NO:12 or a subsequence thereof. In some embodiments, the HBG transgene will be linked to the HBA transgene such that they will both be expressed under the control of the endogenous HBB promoter. In some embodiments, the two transgenes are separated by a cleavage peptide sequence, e.g., a Furin-P2A sequence as shown in SEQ ID NO:13. Without being bound by the following theory, it is believed that an HBG transgene can be beneficial if the herein-described methods cleave both copies of the HBB gene, e.g., if the editing efficiency is particularly high. In that case, the alpha and gamma globin expressed from the transgenes could form fetal hemoglobin instead of adult hemoglobin.

The transgene comprises a functional coding sequence for the alpha globin gene, with optional elements such as introns, WPREs, poly A regions, UTRs (e.g. 5′ or 3′ UTRs). The optional elements can be from any source. In particular embodiments, the transgene comprises a 5′ UTR and/or 3′ UTR from another globin gene, e.g., from HBA1, HBA2, or HBB. For example, in one embodiment, an HBA1 transgene comprises an HBA1 5′ UTR and/or HBA1 3′ UTR. In one embodiment, an HBA1 transgene comprises an HBA2 5′ UTR and/or HBA2 3′ UTR. In one embodiment, an HBA1 transgene comprises an HBB 5′ UTR and/or HBB 3′ UTR. In one embodiment, an HBA2 transgene comprises an HBA1 5′ UTR and/or HBA1 3′ UTR. In one embodiment, an HBA2 transgene comprises an HBA2 5′ UTR and/or HBA2 3′ UTR. In one embodiment, an HBA2 transgene comprises an HBB 5′ UTR and/or HBB 3′ UTR. It will be appreciated that the 5′ and 3′ UTRs need not be from the same source, e.g., an HBA1 transgene can comprise a 5′UTR from HBA1 and a 3′UTR from HBA2 or HBB. In particular embodiments, the coding sequence within the transgene is from HBA1. In particular embodiments, the 5′ and/or 3′ UTRs within the transgene are from HBB or HBA1. In some embodiments, the 5′ UTR comprises the sequence shown as SEQ ID NO:7 or SEQ ID NO:9 or a subsequence thereof, or a sequence comprising at least about 70%, 75%, 80%, 85%, 90%, 95%, 96%, 97%, 98%, 99%, or more homology to SEQ ID NO:7 or SEQ ID NO: 9. In some embodiments, the 3′ UTR comprises the sequence shown as SEQ ID NO:8, SEQ ID NO:10, or SEQ ID NO:11 or a subsequence thereof, or a sequence comprising at least about 70%, 75%, 80%, 85%, 90%, 95%, 96%, 97%, 98%, 99%, or more homology to SEQ ID NO:8, SEQ ID NO: 10, or SEQ ID NO:11.

In some embodiments, the transgene in the homologous repair template is codon-optimized, e.g., comprises at least about 70%, 75%, 80%, 85%, 90%, 95%, or more homology to the corresponding wild-type coding sequence or cDNA, or a fragment thereof.

In particular embodiments, the template further comprises a polyA sequence or signal, e.g., a bovine growth hormone polyA sequence or a rabbit beta-globin polyA sequence, at the 3′ end of the cDNA. In particular embodiments, a Woodchuck Hepatitis Virus Posttranscriptional Regulatory Element (WPRE) is included within the 3′UTR of the template, e.g., between the 3′ end of the coding sequence and the 5′ end of the polyA sequence, so as to increase the expression of the transgene. Any suitable WPRE sequence can be used; See, e.g., Zufferey et al. (1999) J. Virol. 73 (4): 2886-2892; Donello, et al. (1998). J Virol 72:5085-5092; Loeb, et al. (1999). Hum Gene Ther 10:2295-2305; the entire disclosures of which are herein incorporated by reference).

To facilitate homologous recombination, the transgene is flanked within the polynucleotide or donor construct by sequences homologous to the target genomic sequence. In particular, the transgene is flanked by sequences adjacent to the translation start site and to the site of cleavage as defined by the guide RNA. In particular embodiments, the transgene is flanked by one sequence homologous to the region 5′ to the translation start site (i.e., starting at or around the translation start site and running upstream from the start site) and a second sequence homologous to the region 3′ of the site of cleavage (i.e., starting at or around the cleavage site and running downstream from the cleavage site). Accordingly, upon cleavage and repair of the DNA break using the homologous repair template, the entire region of the HBB locus between the translation start site and the site of cleavage is replaced by the HBA transgene, starting with a single CRISPR-Cas9-mediated cleavage event. In particular embodiments, the HBB left homology arm comprises the sequence shown as SEQ ID NO:1 or a subsequence thereof, or to a sequence comprising at least about 70%, 75%, 80%, 85%, 90%, 95%, 96%, 97%, 98%, 99% or more homology to SEQ ID NO:1 or a subsequence thereof. In particular embodiments, the HBB right homology arm comprises the sequence shown as SEQ ID NO:2 or a subsequence thereof, or to a sequence comprising at least about 70%, 75%, 80%, 85%, 90%, 95%, 96%, 97%, 98%, 99% or more homology to SEQ ID NO:2 or a subsequence thereof. In particular embodiments, the transgene replaces the coding sequence of HBB such that its expression is driven by the endogenous HBB promoter.

In some embodiments, a part or a fragment of the target gene is replaced by the transgene. In some embodiments, the whole coding sequence of the target gene is replaced by the transgene. In some embodiments, the coding sequence and regulatory sequences of the transgene is replaced by the transgene. In some embodiments, the target gene sequence replaced by the transgene comprises an open reading frame. In some embodiments, the target gene sequence replaced by the transgene comprises an expression cassette. In some embodiments, the target gene sequence replace by the transgene comprises a sequence that transcribes into a precursor mRNA. In some embodiments, the target gene sequence replaced by the transgene comprises a 5′UTR, one or more introns, one or more exons, and a 3′ UTR.

In some embodiments, the 5′ (or left) homology arm is at least 100 bp, 200 bp, 300 bp, 400 bp, 500 bp, 600 bp, 700 bp, 800 bp, 900 bp, 1000 bp or more in length. In some embodiments, the, the 5′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 5′ homology arm is at least 400 bp in length. In some embodiments, the 5′ homology arm is at least 500 bp, 600 bp, 700 bo, 800 bp, 900 bp, or 1000 bp in length. In some embodiments, the 5′ homology arm is at least 850 bp in length. In some embodiments, the 5′ homology arm is 400-500 bp. In some embodiments, the 5′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length. In particular embodiments, the 5′ homology arm is about 900 nucleotides in length.

In some embodiments, the 3′ (or right) homology arm is at least 100 bp, 200 bp, 300 bp, 400 bp, 500 bp, 600 bp, 700 bp, 800 bp, 900 bp, 1000 bp or more in length. In some embodiments, the, the 3′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 3′ homology arm is at least 400 bp in length. In some embodiments, the 3′ homology arm is at least 500 bp, 600 bp, 700 bo, 800 bp, 900 bp, or 1000 bp in length. In some embodiments, the 3′ homology arm is at least 850 bp in length. In some embodiments, the 3′ homology arm is 400-500 bp. In some embodiments, the 3′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length. In particular embodiments, the 3′ homology arm is about 900 nucleotides in length.

Any suitable method can be used to introduce the polynucleotide, or donor construct, into the cell. In particular embodiments, the polynucleotide is introduced using a recombinant adeno-associated viral vector (rAAV). For example, the rAAV can be from serotype 1 (e.g., an rAAV1 vector), 2 (e.g., an rAAV2 vector), 3 (e.g., an rAAV3 vector), 4 (e.g., an rAAV4 vector), 5 (e.g., an rAAV5 vector), 6 (e.g., an rAAV6 vector), 7 (e.g., an rAAV7 vector), 8 (e.g., an rAAV8 vector), 9 (e.g., an rAAV9 vector), 10 (e.g., an rAAV10 vector), or 11 (e.g., an rAAV11 vector). In particular embodiments, the vector is an rAAV6 vector. In some instances, the donor template is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to the HBB locus, and (b) an HBA transgene as described herein. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g. an AAV backbone, to generate virus; (2) arms of homology to the target site of at least 200 bp but ideally at least 400 bp or at least 900 on each side to assure high levels of reproducible targeting to the site (see, Porteus, Annual Review of Pharmacology and Toxicology, Vol. 56:163-190 (2016); which is hereby incorporated by reference in its entirety); (3) a transgene encoding a functional alpha globin protein and capable of expressing the functional alpha globin protein, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb, 5-6 kb, 6-7 kb, 7-8 kb, or larger.

Suitable marker genes are known in the art and include Myc, HA, FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

In some embodiments, the donor template or vector comprises a nucleotide sequence homologous to a fragment of the HBB locus, or a nucleotide sequence is at least 85%, 88%, 90%, 92%, 95%, 98%, or 99% identical to at least 200, 250, 300, 350, 400, 450, 500, 600, 700, 800, 900 or more consecutive nucleotides of the HBB locus.

The inserted construct can also include other safety switches, such as a standard suicide gene into the locus (e.g. iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

The present methods allow for the efficient integration of the donor template at the endogenous HBB locus. In some embodiments, the present methods allow for the insertion of the donor template in 20%, 25%, 30%, 35%, 40%, 45%, 50%, 55%, 60%, 65%, or more cells, e.g., cells from an individual with α-thalassemia. The methods also allow for high levels of expression of the encoded protein in cells, e.g., cells from an individual with α-thalassemia, with an integrated transgene, e.g., levels of expression that are at least about 1%, 2%, 3%, 4%, 5%, 6%, 7%, 8%, 9%, 10%, 11%, 12%, 13%, 14%, 15%, 16%, 17%, 18%, 19%, 20%, or more relative to the expression level in healthy control cells. The levels of expression obtained using the present methods are sufficient to attain transfusion-independence, which is the hallmark of disease correction.

In some embodiments, the CRISPR-mediated systems as described herein (e.g., comprising a guide RNA, RNA-guided nuclease, and homologous repair template) are assessed in primary HSPCs, e.g., as derived from mobilized peripheral blood or from cord blood. In such embodiments, the HSPCs can be WT primary HSPCs (e.g., for initial testing of the system) or from patient-derived HSPCs (e.g., for pre-clinical in vitro testing). In some embodiments, the present disclosure provides an isolated host cell with an integrated HBA (i.e., HBA1 or HBA2) transgene as described herein integrated at the HBB locus. In some embodiments, the isolated host cell is from a patient with alpha thalassemia.

### 5. Methods of Treatment

Following the integration of the transgene into the genome of the HSPC and confirming expression of the encoded therapeutic protein, a plurality of modified HSPCs can be reintroduced into the subject. In one embodiment, the HSPCs are introduced by intrafemoral injection, such that they can populate the bone marrow and differentiate into, e.g., red blood cells. In some embodiments, the HSPCs are induced to differentiate into red blood cells in vitro, and the modified red blood cells are then re-introduced into the subject.

Disclosed herein, in some embodiments, are methods of treating a genetic disorder, e.g., α-thalassemia in an individual in need thereof, the method comprising providing to the individual a protein replacement therapy using the genome modification methods disclosed herein. In some instances, the method comprises a modified host cell ex vivo, comprising a functional transgene, e.g., HBA transgene, integrated at the HBB locus, wherein the modified host cell expresses the encoded protein which is deficient in the individual, thereby treating the genetic disorder in the individual.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals.

In some embodiments, a pharmaceutical composition comprising a modified autologous host cell as described herein is provided. The modified autologous host cell is genetically engineered to comprise an integrated transgene at the HBB locus. The modified host cell of the disclosure herein may be formulated using one or more excipients to, e.g.: (1) increase stability; (2) alter the biodistribution (e.g., target the cell line to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously.

In some embodiments, a subject will undergo a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318 (5854) Science 1296-9 (2007); Palchaudari et al., 34 (7) Nature Biotechnology 738-745 (2016); Chhabra et al., 10:8 (351) Science Translational Medicine 351ra105 (2016)) and CAR T-mediated conditioning (see, e.g., Arai et al., 26 (5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the disorder, e.g., β-thalassemia. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cells of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of β-thalassemia or other genetic disorder is also encompassed by the disclosure.

The present disclosure also contemplates kits comprising compositions or components of the present disclosure, e.g., sgRNA, Cas9, RNPs, i53, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

### 6. Examples

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Gene Replacement of β-Globin with α-Globin Restores Hemoglobin Balance in α-Thalassemia-Derived Hematopoietic Stem and Progenitor Cells

### Introduction

We have developed a novel, potentially curative treatment strategy for alpha-thalassemia by using CRISPR-mediated genome editing to integrate a full-length alpha-globin transgene at the start site of the beta-globin locus. This achieves erythroid-specific expression of our integrated alpha-globin and, by simultaneously increasing levels of alpha-globin while decreasing levels of beta-globin, may normalize the pathogenic globin-chain imbalance.

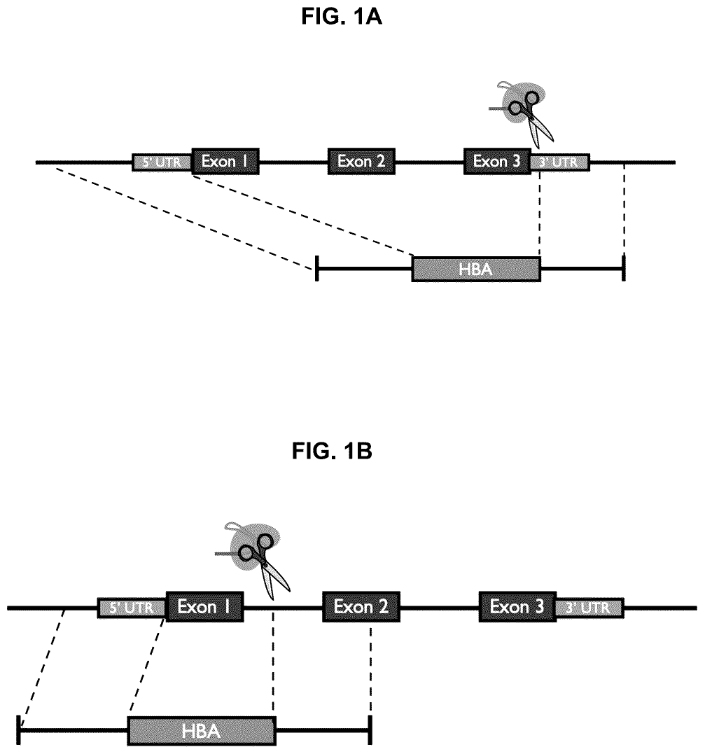

We previously demonstrated the ability to replace large segments of the genome with custom integrations using CRISPR/AAV6-mediated genome editing (Cromer, et al. Nature Medicine, 2021). This was achieved when cleavage was initiated by a single Cas9 gRNA followed by transduction with an AAV6 DNA repair template with a right homology arm that corresponds to the ˜900 bp immediately downstream of the Cas9 cut site and a left homology arm that is split off from the cut site to correspond to the ˜900 bp immediately upstream of the start codon of the endogenous gene (FIG. 1). We found this strategy was capable of mediating a high frequency of recombination in primary human CD34+ HSPCs.

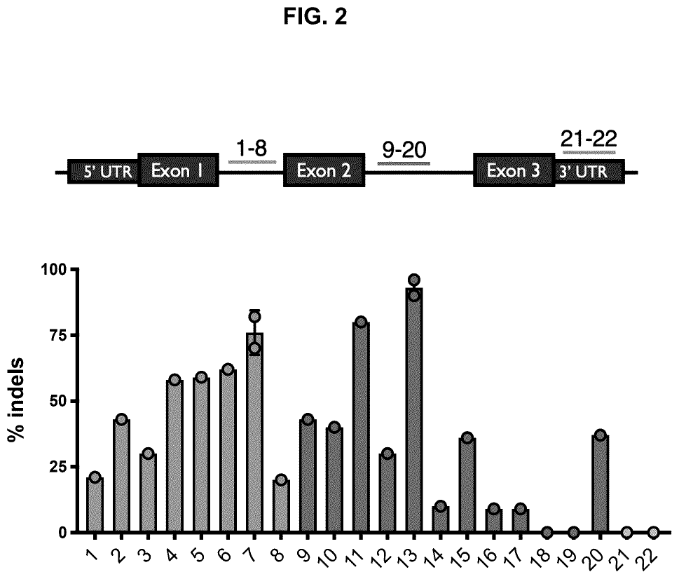

Custom Cas9 gRNAs were designed using a novel bioinformatic workflow, and the gRNAs were ordered from Synthego. The gRNAs were screened for their efficacy in the human HUDEP-2 cell line by pre-complexing Cas9 protein with each respective gRNA and delivering these complexes by electroporation. 2-4d post-targeting, genomic DNA was harvested and subjected to PCR amplification of the region surrounding the expected cut site and resulting Sanger sequences were analyzed for indel frequencies using the online ICE tool from Synthego (FIG. 2).

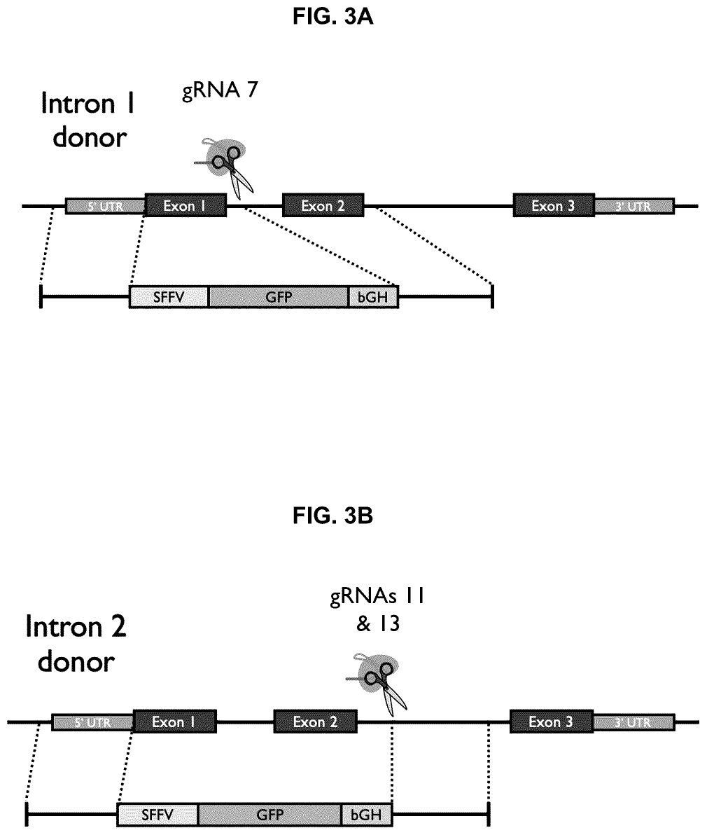

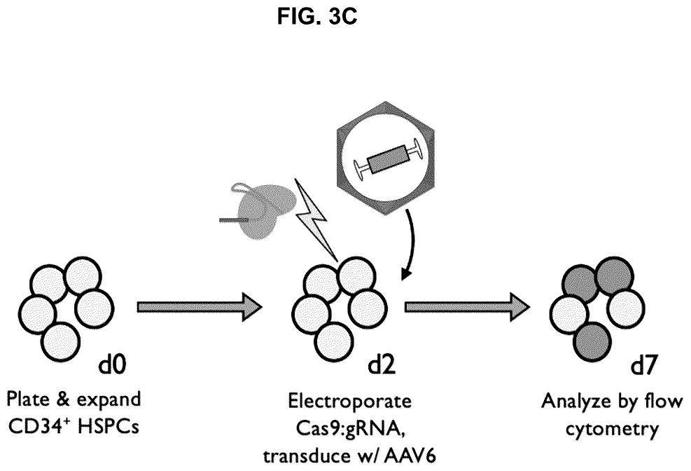

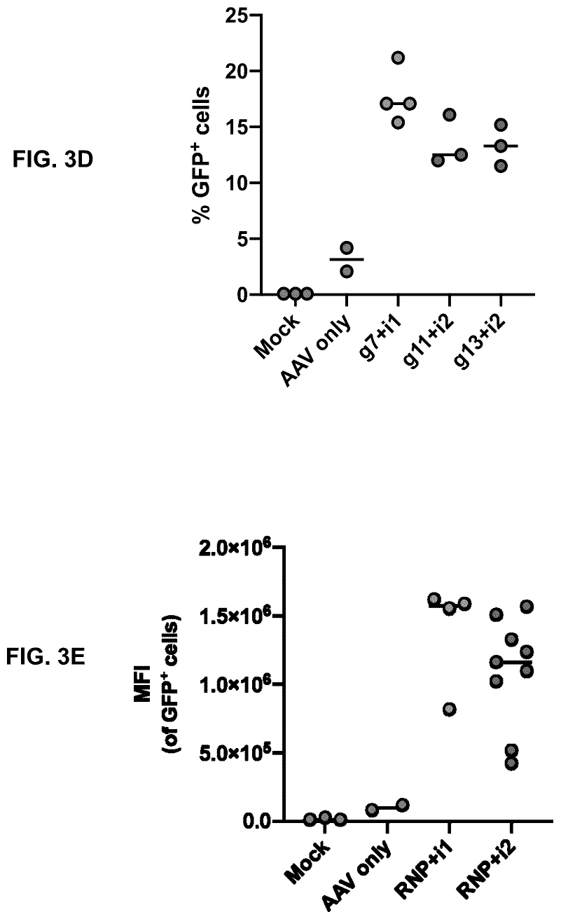

We designed, cloned, produced, and purified AAV6 DNA repair templates with right homology arm that corresponds to the ˜900 bp immediately downstream of the Cas9 cut site and a left homology arm that is split off from the cut site to correspond to the ˜900 bp immediately upstream of the start codon of the endogenous gene (FIGS. 3A and 3B). We then plated and expanded primary human CD34+-enriched HSPCs in HSC expansion media as before (Bak, et al. Nature Protocols, 2016) (FIG. 3C). Two days later, we delivered high-fidelity Cas9 protein pre-complexed with either sgRNA 7, 11, or 13 via electroporation, then immediately transduced HSPCs with the AAV6 DNA repair donor corresponding to intron 1 or intron 2 integration schemes. Five days later, the percentage of GFP+ cells was determined by flow cytometry (FIG. 3D). The intron 1 integration scheme was found to be most efficient and yielded higher mean fluorescence intensities (MFIs) per edited cell than either of the intron 2 integration schemes (FIG. 3E). We proceeded to further optimize our intron 1 integration strategy (using sgRNA 7 and corresponding homology arms).

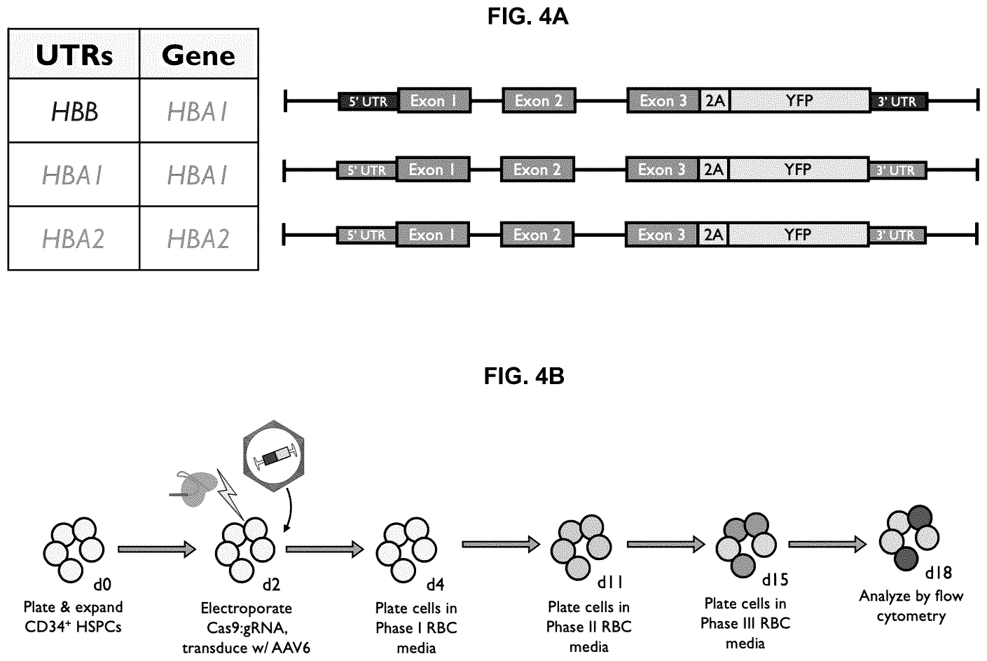

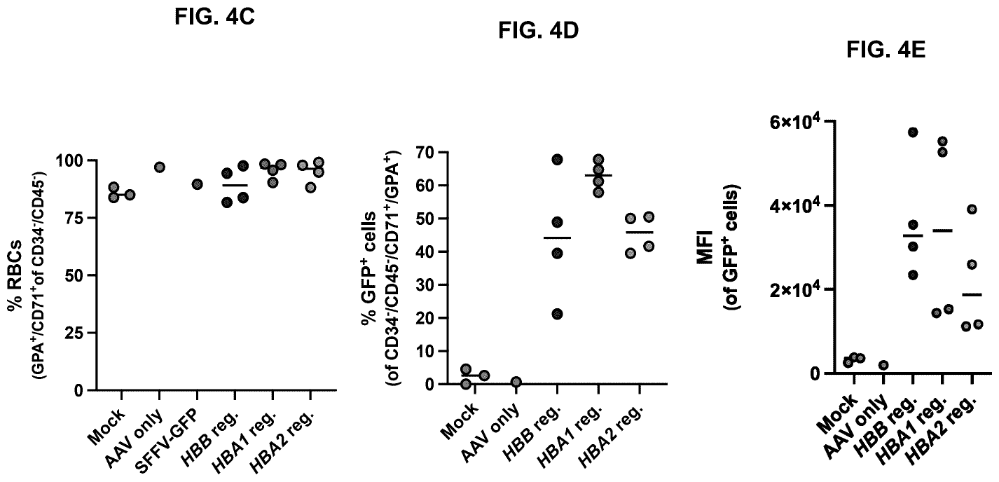

To determine the expression profile when custom transgenes are integrated at the start codon of the endogenous HBB gene, as well as the effect of regulatory regions on editing frequency and expression level, we designed, cloned, produced, and purified promoterless HBA-2A-YFP integration cassettes packaged in AAV6 vectors (FIG. 4A). We then plated, expanded, and edited primary human CD34+ HSPCs as before (9) using sgRNA 7 and transduction with the promoterless repair vectors displayed (FIG. 4B). Because we would not expect HBB to be expressed until HSPCs have begun erythroid differentiation, we used a three-liquid phase erythroid differentiation protocol and measured fluorescence by flow cytometry at day 18 post-editing. These data indicate that erythroid differentiation is unperturbed by our various editing strategies compared to controls. We also found that HBA1 transgene and UTRs yielded the most consistently high editing frequencies and HBB and HBA1 UTRs achieved the highest MFI per edited cell (FIGS. 4C-4E).

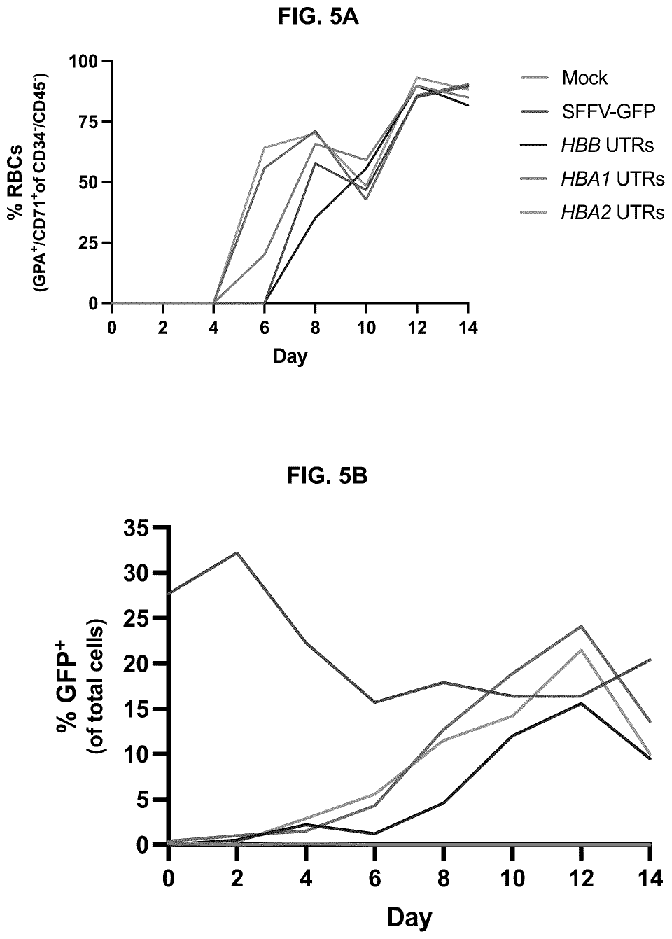

We also tracked expression kinetics by measuring fluorescence by flow cytometry during the course of erythroid differentiation. We found that all editing conditions differentiate at approximately the same rate, and that fluorescence from promoterless HBA-2A-YFP constructs begins to occur around day 6 and plateaus around day 12 (FIG. 5A-5B).

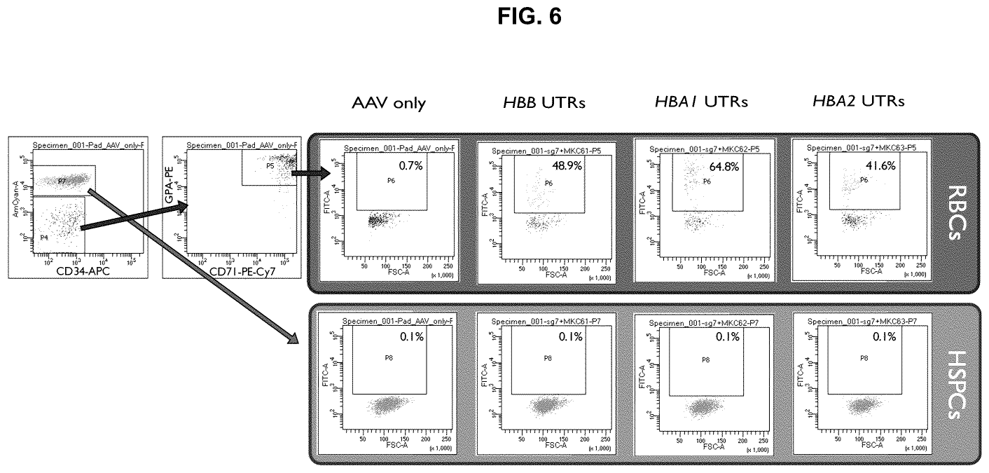

To further confirm that we have achieved erythroid-specific expression that is not leaky, we plotted % of YFP+ cells at day 18 post-editing (at the end of erythroid differentiation) and found no fluorescence above background in CD45+/CD71−/GPA−HSPCs, but a high degree of fluorescence in CD45−/CD71+/GPA+ red blood cells (FIG. 6).

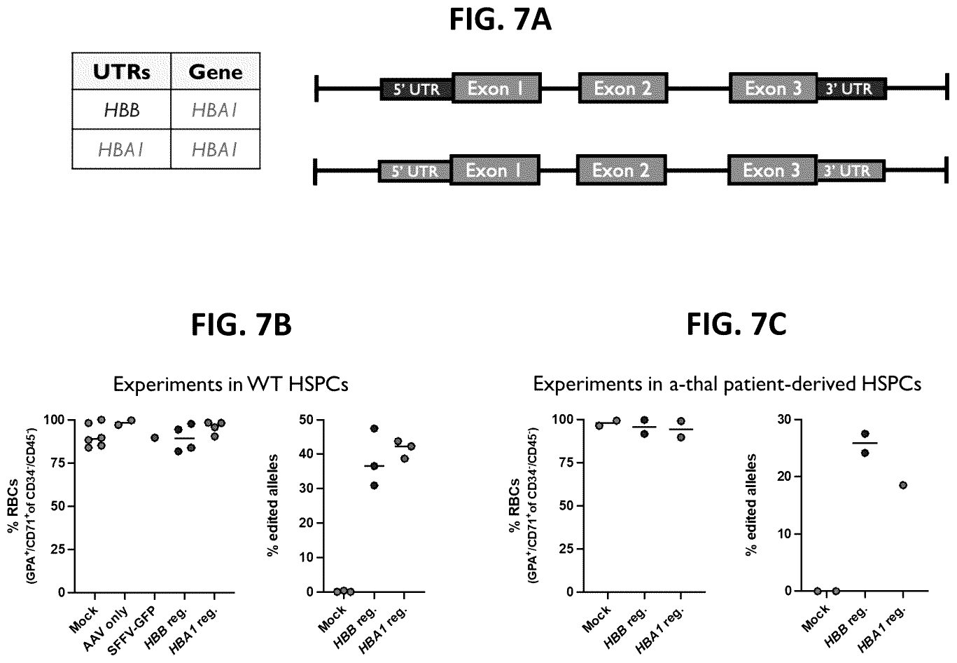

Because of these data, we believe we have identified an erythroid-specific safe harbor site for integration of an alpha-thalassemia correction vector. We designed promoterless HBA1 AAV6 repair templates with HBB and HBA1 UTRs, but with the 2A-YFP removed (FIG. 7A). These translational vectors were first tested in WT primary human CD34+ HSPCs, and they showed an ability to efficiently integrate (FIG. 7B). We then tested these vectors in alpha thalassemia patient-derived CD34+ HSPCs, and they showed an ability to allow normal RBC development and to facilitate high frequency editing of the intended construct (FIG. 7C). mRNA and protein assays are used to ensure that edited cells are able to restore adult hemoglobin tetramers and normalize the alpha-globin: beta-globin chain imbalance in the context of alpha-thalassemia.

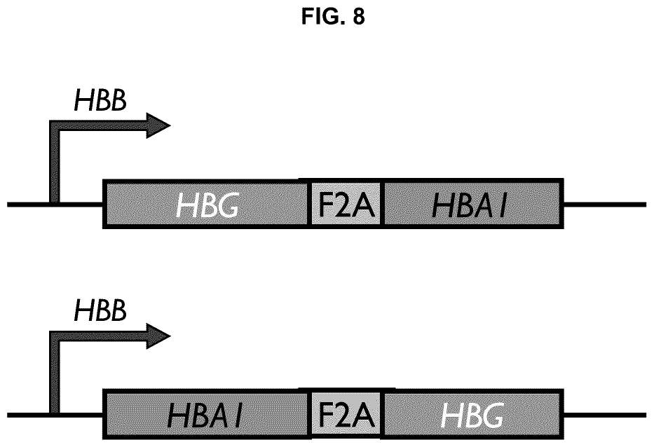

In addition to the vectors described above, we are also designing cassettes that will integrate HBG1 (gamma-globin) along with our HBA1 transgene. These vectors will use sgRNA 7, intron 1 homology arms, and HBA1 UTRs (FIG. 8). Linking these two transgenes will be a Furin-P2A (F2A) cleavage peptide sequence to minimize the possibility that the corresponding cleavage tails at the C-terminus and N-terminus of the two transgenes will disrupt hemoglobin tetramer formation. The reasoning for this approach is that this would minimize the possibility that a high degree of bi-allelic editing would cure alpha-thalassemia, but cause beta-thalassemia (by knocking out both endogenous copies of HBB). We decided to use HBG1 instead of HBB due to complications arising from homology of an HBB transgene cassette integrated at the endogenous HBB locus. The integration of HBG1 is therefore expected to increase levels of fetal hemoglobin, rather than adult hemoglobin tetramers.

### Materials and Methods

### AAV6 Vector Design, Production, and Purification

All AAV6 vectors were cloned into the pAAV-MCS plasmid (Agilent Technologies, Santa Clara, CA, USA), which contains inverted terminal repeats (ITRs) derived from AAV2. Gibson Assembly Mastermix (New England Biolabs, Ipswich, MA, USA) was used for the creation of each vector as per manufacturer's instructions. Few modifications were made to the production of AAV6 vectors as described¹. 293T cells (Life Technologies, Carlsbad, CA, USA) were seeded in ten 15 cm² dishes with 17×10⁶ cells per plate. 24h later, each dish was transfected with a standard polyethylenimine (PEI) transfection of 6 μg ITR-containing plasmid and 22 μg pDGM6 (gift from David Russell, University of Washington, Seattle, WA, USA), which contains the AAV6 cap genes, AAV2 rep genes, and Ad5 helper genes. After a 48-72h incubation, cells were purified using AAVPro Purification Kits (All Serotypes) (Takara Bio USA, Mountain View, CA, USA) as per manufacturer's instructions. AAV6 vectors were titered using ddPCR to measure number of vector genomes as previously described².

### Culturing of CD34⁺ HSPCs

Human CD34⁺ HSPCs were cultured as previously described³⁻⁸. CD34⁺ HSPCs were sourced from fresh cord blood (generously provided by Binns Family program for Cord Blood Research), frozen cord blood and Plerixafor- and/or G-CSF-mobilized peripheral blood (AllCells, Alameda, CA, USA and STEMCELL Technologies, Vancouver, CanadaCD34⁺ HSPCs were cultured at 1×10⁵ cells/mL in StemSpan SFEM II (STEMCELL Technologies, Vancouver, Canada) base medium supplemented with stem cell factor (SCF) (100 ng/mL), thrombopoietin (TPO) (100 ng/mL), FLT3-ligand (100 ng/mL), IL-6 (100 ng/mL), UM171 (35 nM), 20 mg/mL streptomycin, and 20U/mL penicillin. The cell incubator conditions were 37° C., 5% CO2, and 5% O₂.

### Genome Editing of CD34⁺ HSPCS

Chemically modified Cas9 sgRNAs were purchased from Synthego (Menlo Park, CA, USA) and TriLink BioTechnologies (San Diego, CA, USA) and were purified by high-performance liquid chromatography (HPLC). The sgRNA modifications added were the 2′-O-methyl-3′-phosphorothioate at the three terminal nucleotides of the 5′ and 3′ ends described previously⁹. The target sequences for sgRNAs were as follows: sg7: 5′-GGGTGGGAAAATAGACCAAT-3′; sg11: 5′-TATGGTTAAGTTCATGTCAT-3′; and sg13: 5′-TAGGAAGGGGATAAGTAACA-3′. All hi-fidelity variant10 Cas9 protein (SpyFi) used was purchased from Aldevron, LLC (Fargo, ND, USA). The RNPs were complexed at a Cas9: sgRNA molar ratio of 1:2.5 at 25° C. for 10 min prior to electroporation. CD34+ cells were resuspended in P3 buffer (Lonza, Basel, Switzerland) with complexed RNPs and electroporated using the Lonza 4D Nucleofector (program DZ-100). Cells were plated at 1×10⁵ cells/mL following electroporation in the cytokine-supplemented media described previously. Immediately following electroporation, AAV6 was supplied to the cells at 5×10³ vector genomes/cell based on titers determined by ddPCR.

### Gene Targeting Analysis by Flow Cytometry

For targeting analysis by flow cytometry, CD34⁺ HSPCs were harvested at d5 and erythrocytes at d16 post-targeting. Cells were analyzed for viability using Ghost Dye Red 780 (Tonbo Biosciences, San Diego, CA, USA) and reporter expression was assessed using either the FACS Aria II (BD Biosciences, San Jose, CA, USA). The data was subsequently analyzed using FlowJo (FlowJo LLC, Ashland, OR, USA).

In Vitro Differentiation of CD34⁺ HSPCs into Erythrocytes

Following targeting, HSPCs derived from healthy, SCD, or β-thalassemia patients were cultured for 14-16d at 37° C. and 5% CO2 in SFEM II medium (STEMCELL Technologies, Vancouver, Canada) as previously described^{11, 12}. SFEMII base medium was supplemented with 100U/mL penicillin-streptomycin, 10 ng/mL SCF, 1 ng/ml IL-3 (PeproTech, Rocky Hill, NJ, USA), 3U/mL erythropoietin (eBiosciences, San Diego, CA, USA), 200 μg/mL transferrin (Sigma-Aldrich, St. Louis, MO, USA), 3% antibody serum (heat-inactivated from Atlanta Biologicals, Flowery Branch, GA, USA), 2% human plasma (umbilical cord blood), 10 μg/mL insulin (Sigma-Aldrich, St. Louis, MO, USA) and 3U/mL heparin (Sigma-Aldrich, St. Louis, MO, USA). In the first phase, d 0-7 (day zero being 2d post-targeting) of differentiation, cells were cultured at 1×10⁵ cells/mL. In the second phase, d7-10, cells were maintained at 1×10⁵ cells/mL, and IL-3 was removed from the culture. In the third phase, d11-16, cells were cultured at 1×10⁶ cells/mL, and transferrin was increased to 1 mg/mL within the culture medium.

### Immunophenotyping of Differentiated Erythrocytes

HSPCs subjected to the above erythrocyte differentiation were analyzed at d14 for erythrocyte lineage-specific markers using a FACS Aria II (BD Biosciences, San Jose, CA, USA). Edited and non-edited cells were analyzed by flow cytometry using the following antibodies: hCD45 V450 (HI30; BD Biosciences, San Jose, CA, USA), CD34 APC (561; BioLegend, San Diego, CA, USA), CD71 PE-Cy7 (OKT9; Affymetrix, Santa Clara, CA, USA), and CD235a PE (GPA) (GA-R2; BD Biosciences, San Jose, CA, USA).

### REFERENCES

1. Khan, I. F., Hirata, R. K. & Russell, D. W. AAV-mediated gene targeting methods for human cells. Nat Protoc 6, 482-501 (2011).
2. Aurnhammer, C. et al. Universal real-time PCR for the detection and quantification of adeno-associated virus serotype 2-derived inverted terminal repeat sequences. Hum Gene Ther Methods 23, 18-28 (2012).
3. Dever, D. P. et al. CRISPR/Cas9 beta-globin gene targeting in human haematopoietic stem cells. Nature 539, 384-389 (2016).
4. Charlesworth, C. T. et al. Priming Human Repopulating Hematopoietic Stem and Progenitor Cells for Cas9/sgRNA Gene Targeting. Mol Ther Nucleic Acids 12, 89-104 (2018).
5. Bak, R. O., Dever, D. P. & Porteus, M. H. CRISPR/Cas9 genome editing in human hematopoietic stem cells. Nat Protoc 13, 358-376 (2018).
6. Bak, R. O. et al. Multiplexed genetic engineering of human hematopoietic stem and progenitor cells using CRISPR/Cas9 and AAV6. Elife 6 (2017).
7. Cromer, M. K. et al. Global Transcriptional Response to CRISPR/Cas9-AAV6-Based Genome Editing in CD34 (+) Hematopoietic Stem and Progenitor Cells. Mol Ther 26, 2431-2442 (2018).
8. Bak, R. O. & Porteus, M. H. CRISPR-Mediated Integration of Large Gene Cassettes Using AAV Donor Vectors. Cell Rep 20, 750-756 (2017).
9. Hendel, A. et al. Chemically modified guide RNAs enhance CRISPR-Cas genome editing in human primary cells. Nat Biotechnol 33, 985-989 (2015).
10 Vakulskas, C. A. et al. A high-fidelity Cas9 mutant delivered as a ribonucleoprotein complex enables efficient gene editing in human hematopoietic stem and progenitor cells. Nat Med 24, 1216-1224 (2018).
11. Dulmovits, B. M. et al. Pomalidomide reverses gamma-globin silencing through the transcriptional reprogramming of adult hematopoietic progenitors. Blood 127, 1481-1492 (2016).
12. Hu, J. et al. Isolation and functional characterization of human erythroblasts at distinct stages: implications for understanding of normal and disordered erythropoiesis in vivo. Blood 121, 3246-3253 (2013).

### 7. Exemplary Embodiments

Exemplary embodiments provided in accordance with the presently disclosed subject matter include, but are not limited to, the claims and the following embodiments:

Embodiment 1: a method of genetically modifying a hematopoietic stem and progenitor cell (HSPC) from a subject, the method comprising: introducing into the HSPC a guide RNA comprising a sequence that hybridizes to an intron of an HBB gene sequence, an RNA-guided nuclease, and a donor template comprising a transgene encoding an α-globin protein, wherein the donor template comprises a first homology arm located 5′ of the transgene, the first homology arm corresponding to at least 200 nucleotides of the HBB gene sequence beginning at the start codon and continuing upstream thereof, and a second homology arm located 3′ of the transgene, the second homology arm corresponding to at least 200 nucleotides of the HBB gene sequence, beginning at the guide RNA target site and continuing downstream thereof; wherein the RNA-guided nuclease cleaves the intron of the HBB gene sequence in the cell and the transgene is integrated into the cleaved HBB gene sequence; thereby generating a genetically modified HSPC; wherein the integrated transgene results in expression of the α-globin protein in the genetically modified HSPC.

Embodiment 2: the method of embodiment 1, wherein the method further comprises isolating the HSPC from the subject prior to introducing the guide RNA, the RNA-guided nuclease, and the donor template.

Embodiment 3: the method of embodiment 1 or 2, wherein the first homology arm comprises at least about 300, 400, 500, 600, 700, 800, 900, or more nucleotides.

Embodiment 4: the method of any one of embodiments 1 to 3, wherein the first homology arm comprises the nucleotide sequence of SEQ ID NO: 1 or a subsequence thereof, or a sequence comprising at least about 80%, 85%, 90%, 95%, 96%, 97%, 98%, 99%, or more identity to SEQ ID NO:1 or a subsequence thereof.

Embodiment 5: the method of any one of embodiments 1 to 4, wherein the second homology arm comprises at least about 300, 400, 500, 600, 700, 800, 900, or more nucleotides.

Embodiment 6: the method of any one of embodiments 1 to 5, wherein the second homology arm comprises the nucleotide sequence of SEQ ID NO: 2 or a subsequence thereof, or a sequence comprising at least about 80%, 85%, 90%, 95%, 96%, 97%, 98%, 99%, or more identity to SEQ ID NO:2 or a subsequence thereof.

Embodiment 7: the method of any one of embodiments 1 to 6, wherein the intron of the HBB gene sequence is intron 1.

Embodiment 8: the method of embodiment 7, wherein the target sequence of the guide RNA comprises the nucleotide sequence of SEQ ID NO:14.

Embodiment 9: the method of any one of embodiments 1 to 6, wherein the intron of the HBB gene sequence is intron 2.

Embodiment 10: the method of embodiment 9, wherein the target sequence of the guide RNA comprises the nucleotide sequence of SEQ ID NO: 15 or SEQ ID NO:16.

Embodiment 11: the method of any one of embodiments 1 to 10, wherein the guide RNA comprises one or more 2′-O-methyl-3′-phosphorothioate (MS) modifications.

Embodiment 12: the method of embodiment 11, wherein the one or more 2′-O-methyl-3′-phosphorothioate (MS) modifications are present at the three terminal nucleotides of the 5′ and 3′ ends of the guide RNA.

Embodiment 13: the method of any one of embodiments 1 to 12, wherein the RNA-guided nuclease is Cas9.

Embodiment 14: the method of any one of embodiments 1 to 13, wherein the guide RNA and the RNA-guided nuclease are introduced into the HSPC as a ribonucleoprotein (RNP) complex by electroporation.

Embodiment 15: the method of any one of embodiments 1 to 14, wherein the transgene is an HBA1 transgene.

Embodiment 16: the method of embodiment 15, wherein the transgene comprises the nucleotide sequence of SEQ ID NO:4, or a sequence comprising at least about 80%, 85%, 90%, 95%, 96%, 97%, 98%, 99%, or more identity to SEQ ID NO:4.

Embodiment 17: the method of any one of embodiments 1 to 14, wherein the transgene is an HBA2 transgene.

Embodiment 18: the method of embodiment 17, wherein the transgene comprises the nucleotide sequence of SEQ ID NO:5, or a sequence comprising at least about 80%, 85%, 90%, 95%, 96%, 97%, 98%, 99%, or more identity to SEQ ID NO:5.

Embodiment 19: the method of any one of embodiments 1 to 18, wherein the expression of the transgene is driven by an endogenous HBB promoter.

Embodiment 20: the method of any one of embodiments 1 to 19, wherein the transgene comprises a 5′ UTR derived from the HBB gene.

Embodiment 21: the method of embodiment 20, wherein the 5′ UTR comprises the nucleotide sequence of SEQ ID NO:7.

Embodiment 22: the method of any one of embodiments 1 to 19, wherein the transgene comprises a 5′ UTR derived from the HBA1 or HBA2 gene.

Embodiment 23: the method of embodiment 22, wherein the 5′ UTR comprises the nucleotide sequence of SEQ ID NO:9.

Embodiment 24: the method of any one of embodiments 1 to 23, wherein the transgene comprises a 3′ UTR derived from the HBB gene.

Embodiment 25: the method of embodiment 24, wherein the 3′ UTR comprises the nucleotide sequence of SEQ ID NO:8.

Embodiment 26: the method of any one of embodiments 1 to 23, wherein the transgene comprises a 3′ UTR derived from the HBA1 or HBA2 gene.

Embodiment 27: the method of embodiment 26, wherein the 3′ UTR comprises the nucleotide sequence of SEQ ID NO: 10 or SEQ ID NO:11.

Embodiment 28: the method of any one of embodiments 1 to 27, wherein the donor template is introduced into the HSPC using a recombinant adeno-associated virus (rAAV) vector.

Embodiment 29: the method of embodiment 28, wherein the rAAV vector is a AAV6 vector.

Embodiment 30: the method of any one of embodiments 1 to 29, wherein the HSPC comprises an HBA1 or HBA2 gene that comprises a mutation or deletion as compared to a wild type HBA1 or HBA2 gene.

Embodiment 31: the method of embodiment 30, wherein the mutation is causative of a disease.

Embodiment 32: the method of embodiment 31, wherein the disease is alpha-thalassemia.

Embodiment 33: the method of any one of embodiments 1 to 32, wherein the method increases the level of adult hemoglobin tetramers in the HSPC as compared to prior to introduction of the guide RNA, the RNA-guided nuclease, and the donor template.

Embodiment 34: the method of any one of embodiments 1 to 33, wherein the subject has alpha-thalassemia, and wherein the genetically modified HSPC is reintroduced into the subject.

Embodiment 35: the method of embodiment 34, wherein the reintroduction of the genetically modified HSPC ameliorates one or more symptoms of the alpha-thalassemia.

Embodiment 36: the method of any one of embodiments 1 to 35, wherein the subject is a human.

Embodiment 37: a genetically modified HSPC comprising an HBA1 or HBA2 transgene integrated in an HBB locus, wherein the genetically modified HSPC is generated using the method of any one of embodiments 1 to 36.

Embodiment 38: the genetically modified HSPC of embodiment 37, wherein the HBA1 or HBA2 transgene has replaced an endogenous HBB coding sequence in the genome of the genetically modified HSPC.

Embodiment 39: a population of HSPCs comprising the genetically modified HSPC of embodiment 37 or 38.

Embodiment 40: a method for treating alpha-thalassemia in a subject in need thereof, the method comprising administering the genetically modified HSPC of embodiment 37 or 38 to the subject, wherein the genetically modified HSPC engrafts in the subject and results in an increased level of adult hemoglobin tetramers in the subject as compared to prior to the administration, thereby treating alpha-thalassemia in the subject.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of genetically modifying a hematopoietic stem and progenitor cell (HSPC) from a subject, the method comprising:
introducing into the HSPC a guide RNA comprising a sequence that hybridizes to an intron of an HBB gene sequence, an RNA-guided nuclease, and a donor template comprising a transgene encoding an α-globin protein, wherein
the donor template comprises a first homology arm located 5′ of the transgene, the first homology arm corresponding to at least 200 nucleotides of the HBB gene sequence beginning at the start codon and continuing upstream thereof, and a second homology arm located 3′ of the transgene, the second homology arm corresponding to at least 200 nucleotides of the HBB gene sequence, beginning at the guide RNA target site and continuing downstream thereof; wherein
the RNA-guided nuclease cleaves the intron of the HBB gene sequence in the cell and the transgene is integrated into the cleaved HBB gene sequence; thereby generating a genetically modified HSPC; wherein
the integrated transgene results in expression of the α-globin protein in the genetically modified HSPC.

2. The method of claim 1, wherein the method further comprises isolating the HSPC from the subject prior to introducing the guide RNA, the RNA-guided nuclease, and the donor template.

3. The method of claim 1, wherein the first homology arm comprises at least about 300, 400, 500, 600, 700, 800, 900, or more nucleotides.

4. The method of claim 1, wherein the first homology arm comprises the nucleotide sequence of SEQ ID NO: 1 or a subsequence thereof, or a sequence comprising at least about 80%, 85%, 90%, 95%, 96%, 97%, 98%, 99%, or more identity to SEQ ID NO:1 or a subsequence thereof.

5. The method of claim 1, wherein the second homology arm comprises at least about 300, 400, 500, 600, 700, 800, 900, or more nucleotides.

6. The method of claim 1, wherein the second homology arm comprises the nucleotide sequence of SEQ ID NO: 2 or a subsequence thereof, or a sequence comprising at least about 80%, 85%, 90%, 95%, 96%, 97%, 98%, 99%, or more identity to SEQ ID NO:2 or a subsequence thereof.

7. The method of claim 1, wherein the intron of the HBB gene sequence is intron 1.

8. The method of claim 7, wherein the target sequence of the guide RNA comprises the nucleotide sequence of SEQ ID NO: 14.

9. The method of claim 1, wherein the intron of the HBB gene sequence is intron 2.

10. The method of claim 9, wherein the target sequence of the guide RNA comprises the nucleotide sequence of SEQ ID NO:15 or SEQ ID NO:16.

11. The method of claim 1, wherein the guide RNA comprises one or more 2′-O-methyl-3′-phosphorothioate (MS) modifications.

12. The method of claim 11, wherein the one or more 2′-O-methyl-3′-phosphorothioate (MS) modifications are present at the three terminal nucleotides of the 5′ and 3′ ends of the guide RNA.

13. The method of claim 1, wherein the RNA-guided nuclease is Cas9.

14. The method of claim 1, wherein the guide RNA and the RNA-guided nuclease are introduced into the HSPC as a ribonucleoprotein (RNP) complex by electroporation.

15. The method of claim 1, wherein the transgene is an HBA1 transgene.

16. The method of claim 15, wherein the transgene comprises the nucleotide sequence of SEQ ID NO:4, or a sequence comprising at least about 80%, 85%, 90%, 95%, 96%, 97%, 98%, 99%, or more identity to SEQ ID NO:4.

17. The method of claim 1, wherein the transgene is an HBA2 transgene.

18. The method of claim 17, wherein the transgene comprises the nucleotide sequence of SEQ ID NO:5, or a sequence comprising at least about 80%, 85%, 90%, 95%, 96%, 97%, 98%, 99%, or more identity to SEQ ID NO:5.

19. The method of claim 1, wherein the expression of the transgene is driven by an endogenous HBB promoter.

20. The method of claim 1, wherein the transgene comprises a 5′ UTR derived from the HBB gene.

21. The method of claim 20, wherein the 5′ UTR comprises the nucleotide sequence of SEQ ID NO:7.

22. The method of claim 1, wherein the transgene comprises a 5′ UTR derived from the HBA1 or HBA2 gene.

23. The method of claim 22, wherein the 5′ UTR comprises the nucleotide sequence of SEQ ID NO:9.

24. The method of claim 1, wherein the transgene comprises a 3′ UTR derived from the HBB gene.

25. The method of claim 24, wherein the 3′ UTR comprises the nucleotide sequence of SEQ ID NO:8.

26. The method of claim 1, wherein the transgene comprises a 3′ UTR derived from the HBA1 or HBA2 gene.

27. The method of claim 26, wherein the 3′ UTR comprises the nucleotide sequence of SEQ ID NO: 10 or SEQ ID NO:11.

28. The method of claim 1, wherein the donor template is introduced into the HSPC using a recombinant adeno-associated virus (rAAV) vector.

29. The method of claim 28, wherein the rAAV vector is a AAV6 vector.

30. The method of claim 1, wherein the HSPC comprises an HBA1 or HBA2 gene that comprises a mutation or deletion as compared to a wild type HBA1 or HBA2 gene.

31. The method of claim 30, wherein the mutation is causative of a disease.

32. The method of claim 31, wherein the disease is alpha-thalassemia.

33. The method of claim 1, wherein the method increases the level of adult hemoglobin tetramers in the HSPC as compared to prior to introduction of the guide RNA, the RNA-guided nuclease, and the donor template.

34. The method of claim 1, wherein the subject has alpha-thalassemia, and wherein the genetically modified HSPC is reintroduced into the subject.

35. The method of claim 34, wherein the reintroduction of the genetically modified HSPC ameliorates one or more symptoms of the alpha-thalassemia.

36. The method of claim 1, wherein the subject is a human.

37. A genetically modified HSPC comprising an HBA1 or HBA2 transgene integrated in an HBB locus, wherein the genetically modified HSPC is generated using the method of claim 1.

38. The genetically modified HSPC of claim 37, wherein the HBA1 or HBA2 transgene has replaced an endogenous HBB coding sequence in the genome of the genetically modified HSPC.

39. A population of HSPCs comprising the genetically modified HSPC of claim 37.

40. A method for treating alpha-thalassemia in a subject in need thereof, the method comprising administering the genetically modified HSPC of claim 37 to the subject, wherein the genetically modified HSPC engrafts in the subject and results in an increased level of adult hemoglobin tetramers in the subject as compared to prior to the administration, thereby treating alpha-thalassemia in the subject.


SEQUENCE LISTING (17 sequences)

SEQ ID NO: 1 - DNA, 900 bp, Homo sapiens
  misc_feature 1..900: note = Description of sequence: HBB left homology arm
  source 1..900: mol_type = genomic DNA
attagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacacc

SEQ ID NO: 2 - DNA, 900 bp, Homo sapiens
  misc_feature 1..900: note = Description of sequence: Right homology arm
  source 1..900: mol_type = genomic DNA
ggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaa

SEQ ID NO: 3 - DNA, 1509 bp, synthetic construct
  misc_feature 1..1509: note = Description of sequence: SFFV-GFP-bGH cassette
  source 1..1509: mol_type = other DNA
attaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggtaccgagctcttcgaaggatccatcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgcc

SEQ ID NO: 4 - DNA, 692 bp, synthetic construct
  misc_feature 1..692: note = Description of sequence: HBA1 transgene
  source 1..692: mol_type = other DNA
atggtgctgtctcctgccgacaagaccaacgtcaaggccgcctggggtaaggtcggcgcgcacgctggcgagtatggtgcggaggccctggagaggtgaggctccctcccctgctccgacccgggctcctcgcccgcccggacccacaggccaccctcaaccgtcctggccccggacccaaaccccacccctcactctgcttctccccgcaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgt

SEQ ID NO: 5 - DNA, 685 bp, synthetic construct
  misc_feature 1..685: note = Description of sequence: HBA2 transgene
  source 1..685: mol_type = other DNA
atggtgctgtctcctgccgacaagaccaacgtcaaggccgcctggggtaaggtcggcgcgcacgctggcgagtatggtgcggaggccctggagaggtgaggctccctcccctgctccgacccgggctcctcgcccgcccggacccacaggccaccctcaaccgtcctggccccggacccaaaccccacccctcactctgcttctccccgcaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctctcagggcagaggatcacgcgggttgcgggaggtgtagcgcaggcggcggctgcgggcctgggccgcactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgt

SEQ ID NO: 6 - DNA, 774 bp, synthetic construct
  misc_feature 1..774: note = Description of sequence: T2A-YFP
  source 1..774: mol_type = other DNA
gagggcaggggcagcctgctgacctgcggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 7 - DNA, 50 bp, Homo sapiens
  misc_feature 1..50: note = Description of sequence: HBB 5' UTR
  source 1..50: mol_type = genomic DNA
acatttgcttctgacacaactgtgttcactagcaacctcaaacagacacc

SEQ ID NO: 8 - DNA, 132 bp, Homo sapiens
  misc_feature 1..132: note = Description of sequence: HBB 3' UTR
  source 1..132: mol_type = genomic DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 9 - DNA, 66 bp, Homo sapiens
  misc_feature 1..66: note = Description of sequence: HBA1/2 5' UTR
  source 1..66: mol_type = genomic DNA
cataaaccctggcgcgctcgcggcccggcactcttctggtccccacagactcagagagaacccacc

SEQ ID NO: 10 - DNA, 111 bp, Homo sapiens
  misc_feature 1..111: note = Description of sequence: HBA1 3'UTR
  source 1..111: mol_type = genomic DNA
gctggagcctcggtggccatgcttcttgccccttgggcctccccccagcccctcctccccttcctgcacccgtacccccgtggtctttgaataaagtctgagtgggcggca

SEQ ID NO: 11 - DNA, 110 bp, Homo sapiens
  misc_feature 1..110: note = Description of sequence: HBA2 3'UTR
  source 1..110: mol_type = genomic DNA
gctggagcctcggtagccgttcctcctgcccgctgggcctcccaacgggccctcctcccctccttgcaccggcccttcctggtctttgaataaagtctgagtgggcagca

SEQ ID NO: 12 - DNA, 1443 bp, synthetic construct
  misc_feature 1..1443: note = Description of sequence: HBG1 transgene
  source 1..1443: mol_type = other DNA
atgggtcatttcacagaggaggacaaggctactatcacaagcctctggggaaaagtcaatgtggaagatgctggaggagaaaccctcggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtgtacccatggacacagcggttcttcgacagcttcggcaacctgtcctctgcctctgccatcatgggaaatcccaaagtcaaggcacacggaaaaaaggtgctgacttccttgggagatgccacaaagcatctcgatgatctcaaaggaacatttgctcagctctccgaactccattgtgataagctccatgtcgaccccgaaaattttaaagtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctcggaaatgtcctcgtgaccgttttggcaatccatttcggaaaagagtttacacctgaggtccaagcttcctggcagaagatggtgactgcagtggccagcgctctctcctccagataccac

SEQ ID NO: 13 - DNA, 78 bp, synthetic construct
  misc_feature 1..78: note = Description of sequence: Furin-P2A cleavage peptide sequence
  source 1..78: mol_type = other DNA
cgggctaagagaggcagcggcgccaccaacttcagcctgctgaagcaggccggcgacgtggaagagaaccctggcccc

SEQ ID NO: 14 - DNA, 20 bp, synthetic construct
  misc_feature 1..20: note = Description of sequence: sg7 Target sequence
  source 1..20: mol_type = other DNA
gggtgggaaaatagaccaat

SEQ ID NO: 15 - DNA, 20 bp, synthetic construct
  misc_feature 1..20: note = Description of sequence: sg11 Target sequence
  source 1..20: mol_type = other DNA
tatggttaagttcatgtcat

SEQ ID NO: 16 - DNA, 20 bp, synthetic construct
  misc_feature 1..20: note = Description of sequence: sg13 Target sequence
  source 1..20: mol_type = other DNA
taggaaggggataagtaaca

SEQ ID NO: 17 - protein, 142 aa, Homo sapiens
  REGION 1..142: note = Description of sequence: alpha globin protein sequence
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR